(19) United States
(12) Patent Application Publication
(10) Pub. No.: US 2025/0385013 A1
(43) Pub. Date: Dec. 18, 2025

(54) Mapping Toxicants

(71) Applicant: ToxiMapp Inc., Dover, DE (US)

(72) Inventors: Deborah Hordon, Beverly, MA (US); Peter Cada, Chapel Hill, NC (US); Kathleen Moriarty, Arlington, MA (US); Lloyd Altman, New York, NY (US)

(21) Appl. No.: 19/236,474

(22) Filed: Jun. 12, 2025

Related U.S. Application Data
(60) Provisional application No. 63/659,202, filed on Jun. 12, 2024.

(51) Int. Cl. G16H 50/80; G06F 16/248; G06F 16/29; G16H 50/70

(52) U.S. Cl. CPC G16H 50/80; G06F 16/248; G06F 16/29; G16H 50/70

(57) ABSTRACT

Systems and methods for mapping toxicants are disclosed. A toxicant database is generated by preparing data retrieved from multiple data sources. Based on a query, which may include a location, the toxicant database is accessed and a map is generated from the toxicant database using at least the location in the query. A report may be generated that provides remediation and mitigation information. The map and the report are presented in a user interface.

### TECHNOLOGICAL FIELD OF THE DISCLOSURE

Embodiments disclosed herein generally relate to mapping toxicants in an environment. More particularly, at least some embodiments relate to systems, hardware, software, computer-readable media, and methods for mapping toxicants and facilitating environmentally-driven health improvement, environmental awareness, remediation and/or the like.

### BACKGROUND

Currently, it is very difficult to understand the risk to human health associated with potential/actual environmental toxicant exposures. Information related to the toxicants in a given area is often difficult to find, understand, may be untrusted, and does not provide a holistic view of the risk. In fact, people are generally unaware of toxicants that may be present in the environment.

The lack of knowledge and information may have negative consequences on their health and the environment. For example, because people are unaware of the toxicants that may be in a given area, they are unable to make informed decisions. The ability to make judicious decisions is further complicated by the fact that data related to toxicants in the environment do not provide sufficient information individually and may be untrustworthy.

### BRIEF DESCRIPTION OF THE DRAWINGS

In order to describe the manner in which at least some of the advantages and features of one or more embodiments may be obtained, a more particular description of embodiments will be rendered by reference to specific embodiments thereof which are illustrated in the appended drawings. Understanding that these drawings depict only typical embodiments and are not therefore to be considered to be limiting of the scope of this disclosure, embodiments will be described and explained with additional specificity and detail through the use of the accompanying drawings, in which:

FIG. 1 discloses aspects of architectures, systems, and methods for mapping toxicants;

FIG. 2 discloses aspects of mapping and reporting toxicants in an environment;

FIG. 3A discloses aspects of an example mapping interface;

FIG. 3B discloses additional aspects of a mapping interface;

FIG. 3C discloses additional aspects of a mapping interface;

FIG. 4 discloses aspects of mapping toxicants;

FIGS. 5A-5D disclose aspects of health related information associated with toxicant related mapping layers;

FIG. 6 discloses aspects of a user interface of toxicant mapping; and

FIG. 7 discloses aspects of a computing device, system, or entity.

### DETAILED DESCRIPTION OF SOME EXAMPLE EMBODIMENTS

Embodiments of the invention relate to presenting environmental and/or toxicant-related information to users via a mapping interface, such as a user interface. The mapping interface allows a user to explore toxicants that may be present in an environment. In addition to providing information regarding toxicants, embodiments of the invention may provide, by way of example only, a user with a threat or risk level, a toxin-intensity rating, address based toxicant information, information for responding to toxicant exposure, and/or information for mitigating or treating exposure to toxicants.

The technology of distributing data to users in an effective manner is challenging. This relates to the fact that users are in multiple different locations, have different technology capabilities and are required to interact with specific data sources. Embodiments of the invention provide a practical manner for users to obtain toxicant-related information. Embodiments of the invention further improve data distribution technologies and provide a way to merge and efficiently distribute, in one example, toxicant exposure information and toxicant remediation or mitigation information. Embodiments of the invention allow remediation actions to be performed more efficiently, in a more targeted manner, and/or to users that have interest.

These improvements allow computing systems to distribute data more effectively and efficiently and more particularly to distributing information to multiple users that may want different views of the underlying data or that may be exposed to different toxicants, different concentrations of toxicants, or the like.

By way of example only and not limitation, toxicants include man-made pollutants and/or other substances that are or may be adverse to human health. By way of example and not limitation, toxicants may include pm 2.5 (particulate pollutant that is 2.5 microns or smaller in size), pm 10 (particulate matter in the air that can irritate eyes, throat, worsen asthma, etc.), ozone, lead, carbon monoxide (CO), sulfur dioxide (SO₂), nitrite, radium, sulfate, nitrite, arsenic, lead, asbestos, VOCs (Volatile Organic Compounds), SVOCS (Semi-volatile organic compounds), PCBs, pesticides including glyphosate, nuclear radiation and the like or combinations thereof.

There are many different types and varieties of toxicants. Embodiments of the invention may identify, by way of example only and not limitation, toxicants related to the air, drinking water, the soil, brownfields, toxic waste sites, contaminated waterbodies, nuclear activities and waste, electricity, EMFs, and the like or combinations thereof.

Embodiments of the invention access multiple toxicant databases that may contain toxicant related data representing all media (e.g., air, water, and land) within a user-specified geographic area and provide output in a map-based user interface. Embodiments of the invention may collect information from users as well. Thus, users may become sources of data that can be incorporated into a toxicant database. In some examples, the system may require user provided data to be verified by a trusted source. For example, a user may collect a sample and have the sample tested by a university, an agency, or the like. The results may be uploaded by the user, via a user interface, and incorporated into the user interface generated by the toxicant system.

In addition, embodiments of the invention are configured to scale as additional sources come online. This may allow various entities (e.g., regional, state, county, city, and town agencies) to become or provide sources of data.

As previously stated, conventional data sets are fragmented, misaligned, and are often limited to a particular media or even a particular toxicant. Further, the manner in which toxicants are measured may vary from one data set to the next. Embodiments of the invention generate a toxicant database that transforms the source data into a user-understandable format that is consistent across different geographies and different data sources.

Example data sources that may be used in embodiments of the invention include, but are not limited to, the following:
1. Radiation and nuclear power plants, waste sites, and disposal routes
2. Hospital nuclear wastes (i.e., radio isotopes and radionuclides)
3. Sealed barrels or containers
4. Pipes with lead or other heavy metals
5. Unexploded ordnances
6. Groundwater Supply Toxicant Potentials (e.g., Arsenic, etc.)
7. Radon and other natural gasses emitted from ground into buildings/structures
8. Airports
9. Golf Courses
10. Power Producers (e.g., Coal Plants, natural gas plants)
11. Pipelines (e.g. Natural Gas, Oil)
12. Fracking Sites
13. Refineries
14. Harmful Algal Blooms
15. Methane leaks (e.g. sources include satellite sensors)
16. Petrol transfer stations (e.g. filling tanker trucks) and gas stations
17. Major power transmission lines, substations, etc
18. Concentrated Animal Feeding Operations
19. Hazardous waste sites
20. Toxic release sites
21. EPA Superfund sites
22. Air toxins
23. Impaired waterbodies
24. Water utility quality
25. Contaminated brownfields
26. Lead pipe location data
27. Chemtrails

The data sources may include sources from different countries, different locations, or the like or combinations thereof. Embodiments of the invention may access these data sources and generate a toxicant database that includes all available toxicants or data from these sources. The toxicant database spans media, toxins (toxicants), mitigations, health impacts, and sources of potential toxins or toxicants.

In another example, embodiments of the invention generate or provide a toxicant exposure risk rating for any given geographic area considering the number and type of toxicants present in all three media types (air, water, land) near the user-selected location (e.g. address, longitude and latitude points).

Often, toxicant exposure risk and/or a response to toxicant exposure is left up to the individual. Embodiments of the invention present a user interface that includes or displays a risk rating for the user to easily interpret based on a complex set of determined variables (e.g., toxicant levels) and algorithms to enable consistent and meaningful classifications in the application across all geographies. In one example, classes may be presented as a range of values (e.g. 1-5), statistically distributing the classification ratings among the specified values. This classification system provides both a source-level analysis of toxicants (i.e., surface waters, drinking water, brownfields, hazardous waste sites, air, nuclear, golf courses, etc.) using specific methods to assess the risk levels for each, as well as an overall risk level considering all toxicant-media combinations.

In another example, advances in research-based standards regarding the exposure zone surrounding a toxicant-media location can be incorporated into embodiments of the invention. For example, any information presented in a user interface may reflect the research-based standards. For example, embodiments of the invention may use distances appropriate to toxicant/media locations to assess human health risk. If research suggests a different distance, this may be reflected in the user interface. In another example, high voltage power lines may have a one-mile exposure zone for electro-magnetic waves, whereas lead in the air may have a five mile exposure zone surrounding its source, and an impaired waterbody may have no exposure zone beyond its shoreline or it may have an expanded buffer zone through the water table. A radius-based area classification algorithm, in one example, is used in the classification risk assessment process. Research may indicate alternate spatial region representations are advisable and the algorithm may adapt to alternate methods to assess areas that could be individual to toxicant and media types, and those varied region area shapes and sizes may be combined in the algorithm to produce improved and more accurate results.

In another example, embodiments of the invention provide dynamic integration of live-streamed weather conditions used to model risk assessment (e.g. air quality, wind direction, flooding, other storm effects, etc.). This could include data from existing services, live sensors (both surface and remotely-sensed), and image-based (e.g., UAV, plane, satellite) including both publicly-available as well as for-purchase data. Embodiments of the invention may also predict potential exposure to toxicants. For example, weather forecasting may be used to predict the spread of an air-borne toxicant. This may further identify locations or areas that may present different risk levels over time. For example, a user may travel to a destination in a manner that accounts for current toxicant levels and/or potential toxicant levels. Toxicant levels at multiple locations (e.g., specific addresses) may be predicted or estimated in advance. As the world's climate changes, too, toxicants in the land and water can be moved around by flooding, for example. An embodiment may include flood-related changes in toxin locations and potential exposures.

In another example, embodiments of the invention may personalize an individual user's exposure risk according to their interactions with the media or domain. For instance, a contaminated body of water could have different risk ratings for users who remain at a distance from that body of water, for users who wade in the water, or those who engage in immersive open water swimming. This interaction may be obtained through one or more methods including user input and activity tracking applications. In addition to user activity, data from sources that track or monitor user-locations over time may also contributed to the toxicant assessment and exposure risk. The assessment may further be contextualized through the inclusion of user-disclosed health conditions or medical monitoring devices that may indicate unique risks relative to specific toxicants (e.g. due to the presence of asthma or diabetes).

In another example, curated research resources may aid users in understanding exposure risks. Embodiments of the invention may generate a summary of curated scientific research findings and recommendations for each toxicant-media-disease relationship. All curated scientific research findings of the toxicant-media-disease relationship may be held or stored in a digital library or location-specific reports available to users.

In another example, embodiments of the invention provide users with a summary of their potential exposures from the past according to where they lived and worked through the years, to the extent that all historic data are available. The temporal range will be variable and driven by user inputs (addresses/locations/dates of previous years). A similar summary may be generated for any period of time (e.g., year, month, day, decade).

In another example, embodiments of the invention integrate existing toxicant-media mitigations/cleanups to improve currency and accuracy of the risk assessment process. Not all mitigations for a specific region are known and integrated into current data sources and interfaces. The mitigations may be sourced from publicly available records at a national or regional level or collected through a crowd sourced input that includes methods to assert a trust level in the crowd sourced data on behalf of the user interpreting the assessment. The trust level may rely upon a verification process by the application through reputable data sources or crowd sourcing methods.

In another example, embodiments of the invention account for regulations by specific municipalities. A municipality may have a specific time period to take some level of action to mitigate the impacts of a particular toxicant. This information can be applied to the combined datasets to aid in the assessment of risk over time for a particular location. As described, there are multiple options that can each be taken separately or in combination to assess risks for a given location and/or a particular user at the location. The analysis may be performed using a multitude of techniques including big data analysis, symantec knowledge graphs, machine learning (e.g. predictive analytics), and other types of artificial intelligence (AI).

In another example, toxicant decay or declination in human health effects over time is determined or used. For example, some toxicants may dissipate or change over time impacting the actual level of risk to an end user. Embodiments of the invention include or interpret the projected time frames or changes that occur over these time frames. This may be attributed to several factors including aging and half-life or dispersion of the toxicant. This could impact user understanding of risk assessment and potential mitigation actions by including such factors (e.g. sediment in a body of water, pollution contaminants in air). Additional examples include knowledge of a garbage dump closing prior to the use of specific toxicants or remediation that has occurred by the municipality to remove toxicant sources (e.g. barrels in a dump, chemicals in water).

In another example, a location-based summary combining government/official toxin data sources with social media sentiment or chatter about environmental risks and observations may be generated. This may also leverage any number of data analysis techniques including various types of AI. Types of crowdsourcing may include integration with social media, platform gathered data, and may include ratings on trust levels as well as frequency of input into the system (e.g. social media listening, disperse sources over multiple social media platforms). Source of chatter will be incorporated into risk assessment, mappings, and the like.

In another example, embodiments of the invention may include generating applicable mitigation actions and health-based recommendations resulting from the analysis. Example results may include answers to questions such as, “What do you do about toxin-related diseases?”, and “What are measures that can be taken to reduce impacts of identified toxins?”. Recommendations may include both conventional and alternative medicine to reduce exposure and prevent disease.

Examples of data sources that may be used in generating a toxicant database may include, but are not limited to:

https://airnow.gov provides a high-level assessment of air quality localized to a zip code.

Another service provides air quality rankings at a city level globally, including the total current amount of pollution as well as a historical view. The service is provided by: https://www.iqair.com/us/earth

The Environmental Working Group, http://www.ewg.org provides access to several focused databases, including tap water, a skin deep database, and information on the “dirty dozen” fruits and vegetables. The tap water database includes information on detected toxicants on a municipality or town level connected to the governing body for the tap water source. The skin de ep database contains assessments of cosmetic products, similar to their product assessments to understand the safety of products in your home.

Toxic sites, https://www.toxicsites.us/ contains a list of toxic sites within 10 states. The toxic sites include information on the contamination source when available as well as a timeline for remediation. The sites included may have been remediated and that is indicated in a chart after clicking on a specific listed contaminated site. The organization and presentation of the data is in contrast to the described invention.

Data basin, https://databasin.org/ hosts a range of information including maps containing the locations of fires with a historical view, and data from specific projects including the inlet habitats after a hurricane. The site also hosts a range of databases including agricultural land use, wind turbine, conservation easements, and protected areas. Each of these are distinct as part of individual projects.

Environmental Defense Fund, https://edf.org is focused on delivering “game changing solutions” to aid in improvements to the environment. While also related to the environment, the focus is very different from the defined invention.

Lightbox, https://www.lightboxre.com/ hosts numerous products including Environmental Data Packages offered for the evaluation of commercial real estate. The data includes information on the availability of utilities to a review of environmental factors for the real estate.

CoreLogic https://corelogic.com provides a combination of data sets to aid in the assessment of property. An example includes a dataset combination to assist in predicting the potential impacts of climate on a particular location to complement the knowledge set for an investment. The focus is market intelligence and includes mortgage estimators and other resources.

ArcGIS Online, https://arcGIS.com offers interactive geospatial maps through a software as a service platform.

Risk Factor by First Street https://riskfactor.com provides information on real estate specific to the potential impacts of events such as flooding, wildfire, wind, air quality, and extreme heat. The focus and analysis of data does not overlap with this invention.

The Environmental Protection Agency (EPA) EnviroAtlas, https://enviroatlas.epa.gov/enviroatlas/interactivemap/, is a resource with over 500 databases that can be applied to maps to view the information in context of a surrounding area. This includes multiple types of toxicants as well as information such as floodplains, percentages of asthma in a region, and protected lands to name a few.

The EPA offers another mix of datasets through https://map22.epa.gov/cimc specific to brownfield sites and includes remediation grants.

The EPA also hosts a site My Waterway, https://mywaterway.epa.gov/ that provides detailed information with linked reports specific to each waterway where a report has been filed. The information is dated to the last inspection uploaded to the database and is currently 1.5 years old for most locations. The report also includes information about wildlife impacts, including fish to understand if toxicants such as PCBs and DTD are a concern.

The EPA hosts numerous databases and offers distinct views into specific categories. Another example is https://geopub.epa.gov/dwwidgetapp/ where similar access as provided in the My Waterway site as well as separate views into assessments on drinking water.

The Air Toxics Screening Assessment is an example of another EPA site, https://www.epa.gov/AirToxScreen focused on air specifically. Data and risk analysis is provided on an annual basis. The information provided is local to a census block and detailed for the exposure risk to specific toxicants analyzed in context of exposure risk to cancers. The application requires the user to be familiar with the area and identifies the specific census block to retrieve a report.

The Enforcement and Compliance History Online (ECHO) application, provided by the EPA, https://echo.epa.gov/facilities/facility-search/results includes sites designated with a key code on maps. Sites are identified that have associated reports with a status symbol related to the report details.

The Tropospheric Emissions: Monitoring of Pollution (TEMPO), https://tempo.si.edu/website application measures pollution for North America using satellite views and light collecting mirrors. This information will result in providing additional layers of information on the current state of pollution where visible from satellite views. This may include forest fire smoke, and other atmospheric impacting pollutants. This application is more akin to a data source to the described invention.

Similar capabilities are available in other regions of the world where air quality assessments are measured and reported. One such example includes this resource in China, https://aqicn.org/city/beijing/ This resource is limited to China and the results are available through the application. Numerous applications are available that report on the impacts of a particular domain, a toxin or set of toxins associated with a source of contamination. Other examples include pesticides, https://water.usgs.gov/nawga/pnsp/usage/maps/show_map.php?year=02&map=GLYPHOSATE&hilo=L&disp=Glyphosate, and even PFAS.

Data sources may not provide data in easily consumable formats. For example, drinking water supply provider locations are provided in one API (Application Programming Interface) proffering whereas the violations of EPA drinking water standards are provided over time via another API. In order to simplify and summarize the data, many current and historic violations for one given location need to be counted and scaled to one number. Embodiments of the invention provide this scaling and provide a summary of all drinking water providers within a given user-provided geographic location. Similar processing and summarization of data is performed for many of the data sources. Embodiments of the invention may recognize and account for data duplication, data redundancy, and the like from different sources.

Artificial Intelligence and machine learning may be incorporated to streamline these processes of data processing and summarization. Additionally, AI may be used to distill and simplify the Peer-review studies of each individually-identified toxicant's human health impacts and how conventional and alternative medicine approaches might mitigate exposures and diseases associated with that particular toxicant. Social media and crowd-sourced input will be leverage and processed with AI as well to keep more local and recent data findings at the forefront of outputs.

Embodiments of the invention, in addition to presenting toxicant-related information in a user interface, may also serve as an input for other applications. For example, the information may be incorporated into a travel-based application to provide travel routes, a tourist application to identify risk. A user may allow personal information (e.g., their personal exposure to toxicants) to be shared with medical persons for health related care.

As previously indicated, the number of toxicants is very large. In one example, embodiments of the invention may classify or group toxicants based on their impact on human health. In one example, toxicants that have similar impact may be grouped together and identified under a selected name. For example, there are many varieties of benzene such as methylbenzene, hydroxbenzene, aminobenzene, benzoic acid, and the like. However, the health impacts (or potential health impacts) such as drowsiness, dizziness, headaches, anemia, and the like are similar. Thus, these toxicants may all be classified as benzene. The user interface may allow a user to drill down to the specific type of benzene, but may be presented initially to a user based on a classification system. The classification system may adapt to media/toxicant classifications.

In another example, toxicants may be mapped to childhood disabilities (e.g., cerebral palsy, autism, down syndrome, vision impairment). Linking toxicants to their health impact may also be illustrated in a user interface. There may be an additional layer on the map of the prevalence of childhood disabilities, for instance, at the county level (such data collected by the CDC).

The Figures are representative of devices, systems, architectures, and/or methods.

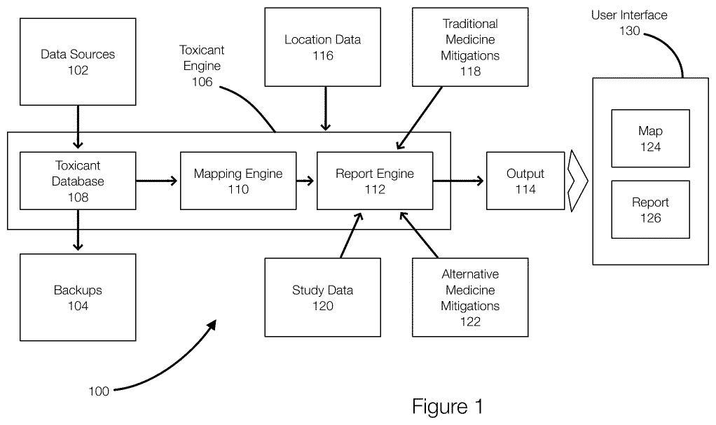

FIG. 1 discloses aspects of systems and methods for mapping toxicants. FIG. 1 illustrates a toxicant system 100, which may be deployed in the cloud, at the edge, in a distributed manner, or the like. The system 100 may include a toxicant engine 106, which may include servers at the edge, in the cloud, distributed geographically, or the like. A client (e.g., a computer, tablet, smartphone) may access the system 100. In response, the toxicant engine 106 may present a user interface 130 (an example of output 114).

The user interface 130 may present different content at different times and may include links to various views or other operations. For example, the user, via a client, may be required to provide credentials or the like. The system 100 may also allow the client to select an operation. For example, the system 100 may allow a user to view a toxicant map generally (e.g., show potential toxicant exposures in a country or state or other geographic area). Alternatively, the user may request information with respect to an environment that includes the location of the user or request information about a specific event.

Thus, the user interface 130 may present various options to the user. FIG. 1 illustrates an example where the output 114 includes data relevant to both a specific event and the user's specific location. In this example, the user interface 130, which is presented at the client in one example, may include a map 124 and a report 126.

The map 124 may include a representation of data retrieved from data sources 102 that has been stored in a toxicant database 108. Thus, the system 100 may retrieve data from multiple data sources, such as those identified previously, and store the retrieved data in a database 180. The data generally includes toxicant related data. Some data may not be relevant and may be discarded. Retrieving the data may include accessing data over APIs or other methods. Backups 104 of relevant data may be generated and stored. This may enable maps to be generated in a time related manner or as a time series and allow a user to view the map 124 at different points of time. This may provide a user with insight regarding toxicant spread in the environment or the like.

The map 124 may be layered. The layers can be viewed together, independently, or the like and may be overlayed onto a geographic map. For example, a user may be interested in water bodies, in populated areas, specific elevations, or the like.

When retrieving data from the data sources 102, the data may be retrieved periodically, in response to queries, in response to environmental events, or the like. The data retrieved from the data sources 102 may be transformed, cleaned, normalized, mapped, or the like to generate the toxicant database 108. This helps ensure that data is normalized or made consistent across different data sources 102. Locations (e.g., latitude/longitude) may be associated with the data if not present in the data. The data may be labeled or processed and prepared for presentation on a device (e.g., a tablet, smartphone, laptop computer, or the like).

The toxicant database 108 may be updated regularly and may be able to provide a time based view. In one example, the toxicant database 108 is accessed based on location data 116 of a client or user. For example, a user may input their location data 116 (or allow their location data 116 to be accessed or, alternatively, input a desired location). This may be done by simply tapping a location on a map, providing a zip code, providing latitude and longitude coordinates, a street address, using global positioning systems, or the like. Many devices allow location to be determined automatically. The location data may also include a range (e.g., a diameter, a defined area, a city, a state, or other geographic boundaries.

With the location data 116, the toxicant database 108 may be accessed and mapping layers and other attributes may be prepared based on the location data 116. The mapping layers may include a layer for various types of mappings. For instance, a user may select a mapping only for hazardous sites, or for drinking water, other waters, soil, brownfields, or the like. These maps may be layered. Attributes (e.g., list of toxicants for each layer) may be displayed as well or may be accessed (e.g., by clicking on a site in the mapping). In addition to the mapping that is presented in the output 114 (e.g., a user interface 130), health impacts (e.g., study data 120), traditional medicine mitigations 118 and alternative medicine mitigations 122 may be included in the outputs 118 and represented in the user interface 130 as a report 126. This allows remedial or mitigation actions to be performed in the event of toxicant exposure.

In one example, the report 126 may be related to the map 124 or to layers of the map 124. In one example, content of the report 126 may change in response to manipulations of the map 124 (e.g., selecting a different layer, a different toxicant, or the like). For example, a user that selects lead may receive a report 126 that reflects lead. Selecting a different toxicant such as radiation, may result in a different report 126. In another example, aspects of the report 126 may be presented selectively according to user input. A user may focus on remedial or mitigation actions, on particular toxicants or groups of toxicants, or the like. User input may determine the information presented in the map 124 and/or the report 126.

In one example, the map 124 and the report 126 may be configured hierarchically such that a user can delve into specific areas and where each layer may include more specific information or data. The hierarchy can be based on geography, toxicant, location, weather, or the like or combinations thereof. For example, a first layer may present multiple toxicants and the user can drill down to a layer that includes a specific toxicant. In another example, a first layer may present or include a map of a city and the user can drill down to a neighborhood in a second layer.

The user interface 130 can be adapted to present other data based on impact on user health, childhood disabilities, or the like. For instance, a user may input selection such that the user interface 130 illustrates toxicants in a particular area with incidents of a particular disease, disorder, or disability. This may be reversed such that the rate of a particular childhood disability may illustrate relevant toxicant levels (current or historical) in a particular area.

In one example, the data sources 102 may, as a result, incorporate data from other sources such as databases that may store disability, disorder, or disease data.

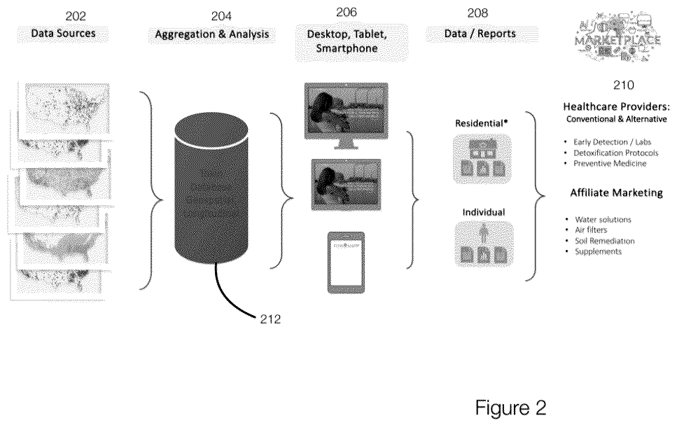

FIG. 2 discloses aspects of mapping and reporting toxicants in an environment. Toxicant data is retrieved or accessed from data sources 202, which are examples of data sources 102. The data is aggregated and analyzed 204 and stored in a toxicant database, such as the toxicant database 108. This allows, by way of example, the data retrieved from the data sources 202 to be presented consistently notwithstanding variations and idiosyncrasies (measurement/collection method, sensor types, region) of the source data 202. The aggregation and analysis 204 may also ensure that the data is associated with geospatial information such as latitude and longitude coordinates. Thus, data from the data sources 102 is prepared and stored in a toxicant database 212, which is an example of the toxicant database 108.

When receiving a location from a user and/or other input (e.g., toxicant, boundaries), a map (or multiple mapping layers) may be generated from the data in the toxicant database 212 (which includes aggregated and analyzed toxicant data) and presented on a device 206. A user may request further data/reports 208, which may be generated based on toxicants identified in the map being generated, health impact studies, treatment options, treatment locations, and the like. The reports 208 or data can be focused on an area or on an individual or have a different focus. For example, if a user's history is available (e.g., previous residences), the cumulative exposures can be generated and presented. This may be generated over a long period of time (e.g., years). In another example, a user's history of locations (e.g., days, weeks) can be used to determine a cumulative exposure to a recent and localized event.

These reports 208 can be used for remediation and mitigation 210, such as early detection, detoxification, preventative medicine. This may also allow a user to be proactive and perform additional remediation or mitigation such as installing water cleaners, air filters, soil cleaning, or the like. In FIG. 2, the reports 208 may include or identify remediations/mitigations 210 such as labs, protocols, preventative medicine and provide links to resources such as water solutions, air filters, and the like.

As illustrated in FIGS. 1 and 2, toxicant mapping enables mitigation and remedial actions to be identified/performed. In one example, mitigation actions typically relate to actions performed to remove or clean up the toxicant. Remedial actions typically relate to actions performed on behalf of a user impacted by exposure to a toxicant, and/or that user's community

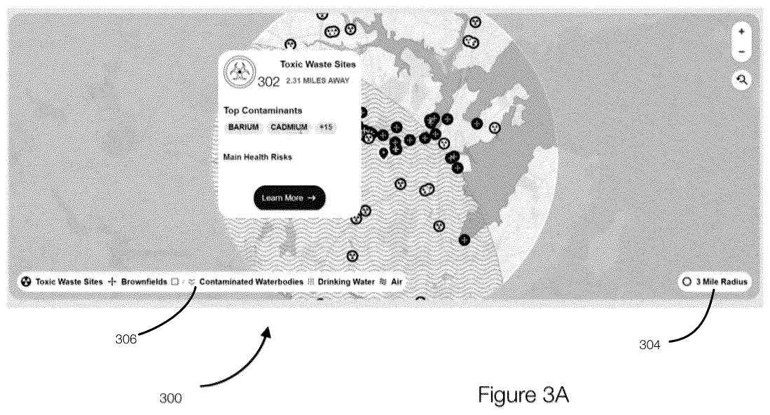

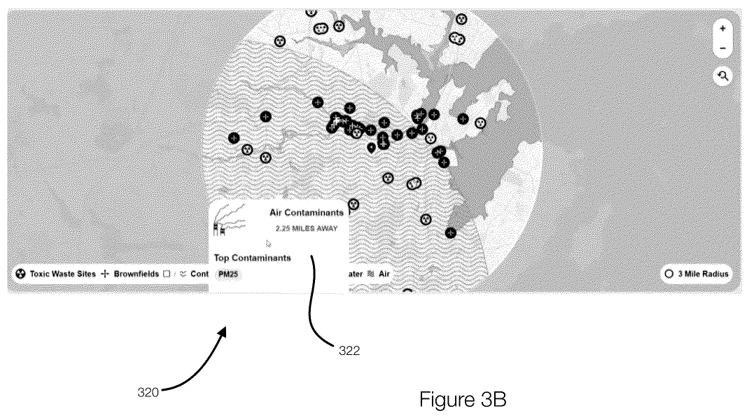

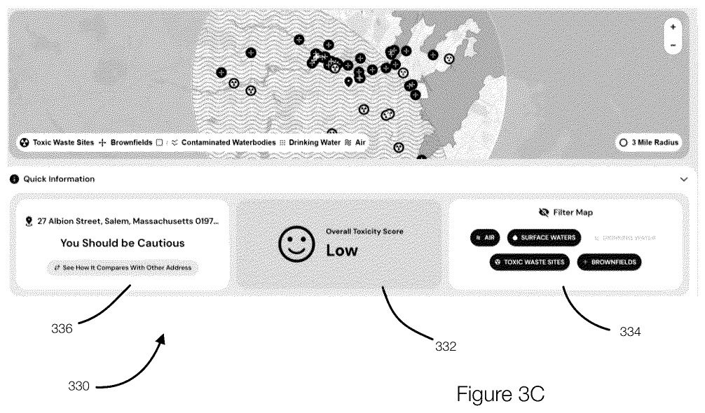

FIG. 3A discloses aspects of an example user interface that may include a mapping and/or reports. The interface 300 illustrates an example mappings. A user may input a location and a map may be generated based on the toxicant database. In this example, a mapping is provided to illustrate toxic waste sites, brownfields, contaminated waterbodies, drinking water, and air. Each different type of toxicant may be represented by a different icon. When an icon is selected for a toxic waste site 302, additional information may be presented such as a list of contaminants, which be ordered according some characteristic such as concentration. The report included in the user interface may also adapt to the user's selection of an icon. A user may be able to learn more about health risks, or the like. The user may also understand their position relative to the position of the selected site. As illustrated, the map can be resized 304 (e.g., by setting a user-proved radius). A default size may be selected.

The user interface 300 can be manipulated to hide/show different layers. Thus, a user only interested in air toxicants may select that layer specifically. For example, the list 306 may be individually selectable such that a user can select a specific layer. The user interface 300 currently illustrates multiple layers simultaneously.

FIG. 3B discloses additional aspects of a mapping interface. The interface 320 illustrates that air contaminants 322 are a certain distance from a user's provided location. Thus, a user with asthma, for example, may be able to avoid areas that may trigger an asthma attack. In one example, the toxicant database may be updated to account for environmental changes (e.g., wind speed, wind direction and other weather conditions). As previously stated, a time lapse view may be viewable. Further a user can update the view to account for most recent data.

FIG. 3C discloses additional aspects of a mapping interface. The interface 330 illustrates a general score 332 may be presented to a user in the interface 330. The score may be low, medium, high, numerical, and have any number of grades. Further, risk scores may be determined for all toxicants collectively or for specific toxicants or map layers such as air, surface waters, toxic waste sites, brownfields, or the like. A user may also be able to compare risk levels for other addresses or locations. This may allow a user to plan a trip by traveling only through low risk areas.

The filter map 334 may allow a user to cause the score 332 to be generated for layers or contaminants selected in the filter 334. Thus, the score 332 may changed based on the selections in the filter map 334. The location filter 336 may allow a user to determine the risk for specific locations. For example, a user may need to travel to a destination. The toxicity score 332 for that location can be identified and/or compared to the score 332 for the user's current location.

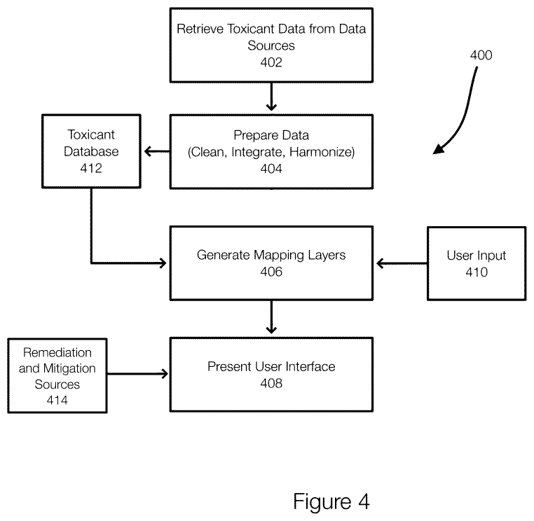

FIG. 4 discloses aspects of methods for mapping toxicants. The method 400 includes retrieving 402 data (e.g., toxicant data) from a plurality of different sources. The retrieved data may be stored. Once data is retrieved and/or stored, the data is prepared 404 for mapping and stored in a toxicant database 412. This includes cleaning the data (e.g., removing extraneous symbols, fields, or the like). This also includes integrating and harmonizing the data. This may also include associating the data with location information. This allows toxicant data to be represented uniformly and consistently regardless of the manner in which the data was collected/generated. This allows for unit conversion, regulations, geographic limitations, or the like to be considered. The data, after being prepared, may be stored in the toxicant database.

Because the data sources may be global in nature, the toxicant database 412 may be able to generate maps and/or reports for any location.

The preparation of the toxicant database 412 may be viewed as an independent method from other aspects of the method 400. Further, the toxicant database 412 may be updated regularly, in response to events (e.g., environmental accidents, weather events, or the like).

Once the toxicant database 412 is prepared, the method 400 is prepared to respond to client or user requests. In one example, a toxicant engine may receive user input 410 (e.g., location data, toxicant, or the like), access the toxicant database 412. Data is retrieved that is relevant to the user input 410. For example, data within a predetermined radius of the provided location may be retrieved and used to generate 406 mapping layers.

Thus, generating 406 the mapping layers may occur in the context of user input 410. Input may include a user's location, another location, one or more toxicant or the like or combinations thereof. The input 410 is used to access the toxicant database 412 to identify toxicant data relevant to the input location. The mapping layers may default to a particular area size (e.g., 5 kilometers). However, the range is adaptable and a user can expand/contract an area of interest. Mapping layers may be generated in different manners. In one example, a mapping layers may be generated based on media (e.g., land, air, water). In another example, mapping layers may be based on category (e.g., toxic waste sites, drinking water, air, soil, waterways, or the like). In another example, mapping layers may be based on toxicant. Thus, a user may view mappings of a toxicant of interest (e.g., pm 2.5).

The mapping layers are presented 408 in a user interface. The user interface may be scalable, allow for mapping layers to be selectively displayed, or the like. The user interface may present a risk level holistically, based on category, based on toxicant, or the like. The user interface may present health risk information, mitigation options, and the like.

In addition, the user interface may include a report from remediation and mitigation sources 414. The content of the report may also be based on the user input 410. For example, the report may include content on lead exposure when the user input 410 specifically identifies lead as a toxicant.

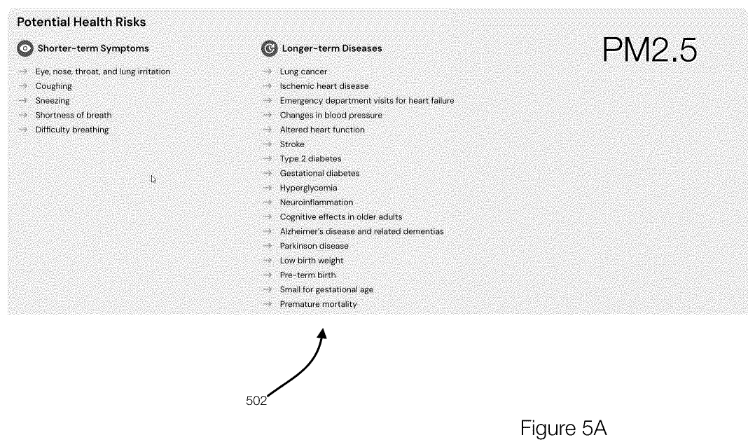

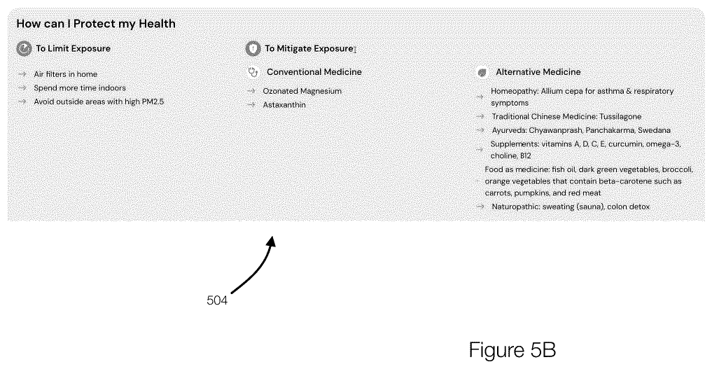

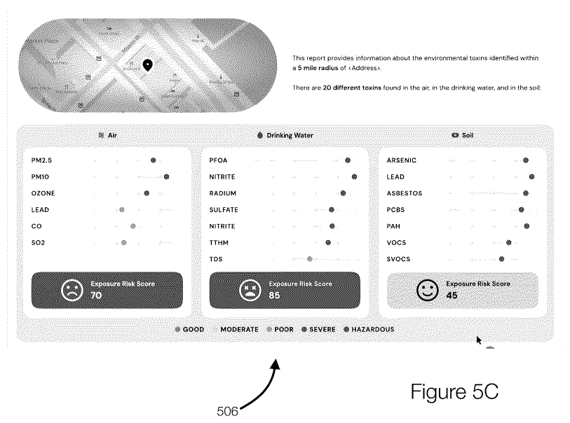

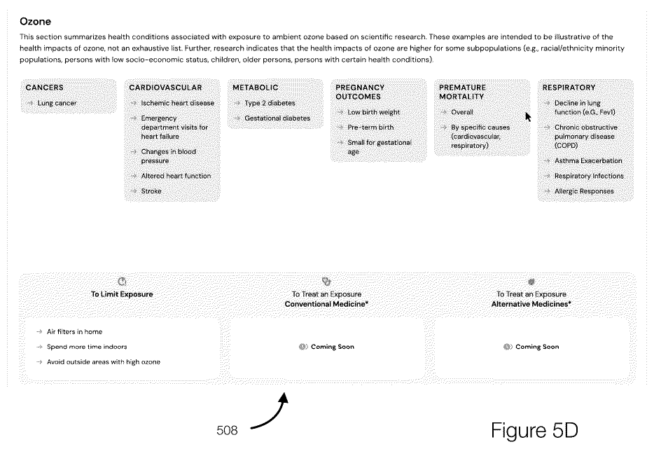

FIGS. 5A-5D disclose aspects of health related information associated with toxicant related mapping layers. The user interface 502 in FIG. 5A illustrates a report that includes potential health risks for a specific toxicant (pm 2.5 in this example). The user interface 504 in FIG. 5B illustrates a report that includes mitigation or remediation actions or recommendations in the event of toxicant exposure. The mitigation or remediation actions may be specific to a selected toxicant.

The interface 506 in FIG. 5C illustrates a report in a user interface that includes a scale of different types of toxicants for different media (air, drinking water, and soil in this example). The interface 506 further illustrates a specific exposure risk score for each media. The interface 506 also identifies a radius in which the toxicants are identified with respect to an input location.

The user interface 508 in FIG. 5D includes a report that presents aspects of a user's health that may be impacted by a toxicant (e.g., Ozone in this example). The interface 508 also provides ways to limit and/or treat toxicant exposure. This demonstrates that the report may pull information from various sources, such as traditional and/or alternative medicine sources.

As illustrated in Figured 5A-5D, the report in the user interface may include a variety of formats and may present data based on user input.

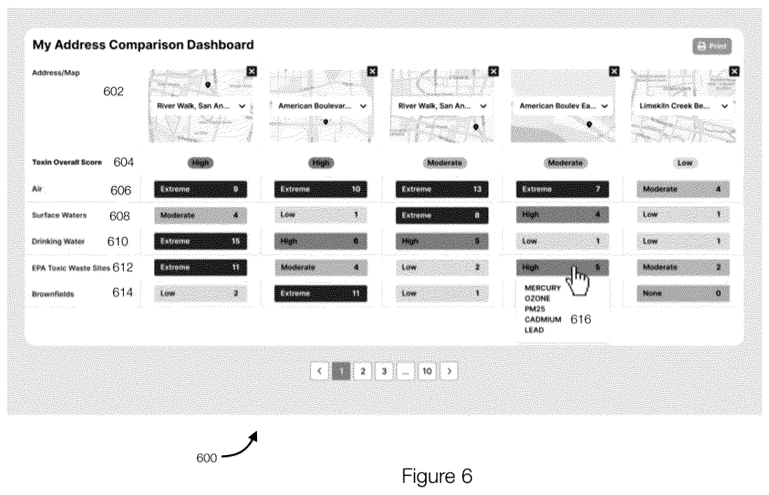

FIG. 6 discloses aspects of a user interface configured to illustrate toxicant data by address or location. The interface 600 is an example of an operation that can be selected by a user. A user may enter multiple address 602 via the user interface 600. The addresses 602 are displayed, in one example, in a map for each address. Using a toxicant database, toxicant data can be retrieved and displayed in the interface 600. In this example, each address 602 is associated with an overall toxin score 604, and individual scores for air 606, surface waters (water bodies) 608, drinking water 610, EPA toxic waste sites 612, and brownfields 614. A user may also be able to drill 616 into each score to identify specific toxicants in one example. In one example, the risk level may be opened to reveal categories or classifications of toxicants. These categories or classifications can be clicked to identify specific toxicants included in the respective classifications.

The address tool in the user interface 600 allows a user to compare various locations, which may or may not be related geographically. For example, a user may select random addresses. Alternatively, a user may select addresses that have a common characteristic (e.g., population density, elevation, weather status, or the like).

One example includes a combination of information on multiple media types (e.g. water, air, land) to enable higher-level assessments. This includes sources of toxicants (e.g. E. coli, pollutants, metals) that currently are, or in the future may leach toxicants, in the surrounding environments (e.g. sealed barrel, storage tanks for gas or oil, lead pipes, unexploded ordinances).

In one example, relying on the combination of one or more media types, a methodology is performed to assess risk for a particular location with some defined proximity for the toxicants that may be exposed to that geographic location. The risk assessment methodology results in a rating at a high-level to the user and optionally in depth information to explain the rating. The rating may be a scale, (e.g. 1-5, low to high). This classification system provides both a source-level analysis of toxicants (i.e., surface waters, drinking water, brownfields, hazardous waste sites, air) using specific algorithms to assess the risk levels for each, as well as an overall risk level considering all toxicant-media combinations.

In one example, for each individual query location or geographical location and media (e.g. air, water), the distance from or known spread of the media (e.g. body of water, underground water tables, air, power lines) to determine the potential toxicant exposures will vary based on known research for the toxicant and media. Within a set radius, the risk may be very low if the field for a particular toxicant is present, but not close enough to have an impact.

The exposure range for a particular toxicant and media within the radius may overlap with only a small and irrelevant portion of the radius and may take on a shape for the area of exposure specific to the media and toxicant.

In one example, a dynamic integration of live-streamed environmental conditions used as factors to augment risk assessment (e.g. air quality, wind direction, storm effects, etc.). This could include data from existing services, live sensors (both surface and remotely-sensed), and image-based (e.g., UAV, plane, satellite) including both publicly-available as well as for-purchase data.

In one example, an individual's exposure to toxicants is calculated based on one or more data sources including user input on interaction with media and/or toxicants, activity tracking applications, a survey, or other tracking information such as information derived from medical device monitoring. This may be factored into the exposure information and risk assessment for a given user in a given location. The individual risk level for a user may be based upon health factors determined in one or more ways including user input or health records.

In one example, the toxicant is identified in each separate interface, with the requirement for the user to perform research on each toxicant. A curated reliable research resources aids users in their individual risk assessments. This curated research may include mitigation measures, best practices on medical advice, and alternative medicine recommendations.

In one example, the ability to profile a user and provide a set of historical potential exposures (e.g. time series graph, summary, timeline) covering residence history, travel, and other potential exposure points that may include daily habits or interactions with points of exposure from leisure, school, or other activities is provided.

In one example, the integration of existing mitigations to aid in the ability to obtain higher fidelity to a risk assessment process. The mitigations may be sourced from publicly available records at a national or regional level and/or collected through a crowd sourced input. Municipality mitigations may be automatically verified or manually depending on the input source (e.g. official/authoritative documents or web sites, authorized personnel, authenticated and authorized users.

In one example, the application of regulations on specific municipalities may impact the assessment of risk and or a timeline wherein the risk level adjusts due to factors such as planned mitigations. As described, there are multiple options that can each be taken separately or in combination to assess risks for a given location and/or a particular user in the location. While each of these are novel and not present in current applications, the combination of each is also novel.

In one example, the risk assessment for a location and/or user may optionally include factors such as toxicants dissipating or changing over time, understanding properties of toxicants and sources of toxicants including half-life. Thus, a projected timeline of toxicants may be provided.

In one example, crowd sourced input on remediation is provided. This may include a method of validation of crowd sourced input through one or more methods (e.g. crowdsourcing, municipality published data) allowing for user assessment of trust or an automated assessment of trust to pre-defined variables. This includes the generation of a location-based summary combining government/official toxin data sources with social media sentiment or chatter about environmental risks and observations. This may also leverage any number of data analysis techniques including various types of AI. Types of crowdsourcing may include integration with social media, platform gathered data, and may include ratings on trust levels as well as frequency of input into the system (e.g. social media listening, disperse sources over multiple social media platforms). Source of chatter will be incorporated into how this novel aspect will be leveraged.

Embodiments, such as the examples disclosed herein, may be beneficial in a variety of respects. For example, and as will be apparent from the present disclosure, one or more embodiments may provide one or more advantageous and unexpected effects, in any combination, some examples of which are set forth below. It should be noted that such effects are neither intended, nor should be construed, to limit the scope of the claims in any way. It should further be noted that nothing herein should be construed as constituting an essential or indispensable element of any embodiment. Rather, various aspects of the disclosed embodiments may be combined in a variety of ways so as to define yet further embodiments.

For example, any element(s) of any embodiment may be combined with any element(s) of any other embodiment, to define still further embodiments. Such further embodiments are considered as being within the scope of this disclosure. As well, none of the embodiments embraced within the scope of this disclosure should be construed as resolving, or being limited to the resolution of, any particular problem(s). Nor should any such embodiments be construed to implement, or be limited to implementation of, any particular technical effect(s) or solution(s). Finally, it is not required that any embodiment implement any of the advantageous and unexpected effects disclosed herein.

It is noted that embodiments disclosed herein, whether claimed or not, cannot be performed, practically or otherwise, in the mind of a human. Accordingly, nothing herein should be construed as teaching or suggesting that any aspect of any embodiment could or would be performed, practically or otherwise, in the mind of a human. Further, and unless explicitly indicated otherwise herein, the disclosed methods, processes, and operations, are contemplated as being implemented by computing systems that may comprise hardware and/or software. That is, such methods processes, and operations, are defined as being computer-implemented.

The following is a discussion of aspects of example operating environments for various embodiments. This discussion is not intended to limit the scope of the claims or this disclosure, or the applicability of the embodiments, in any way.

In general, embodiments may be implemented in connection with systems, software, and components, that individually and/or collectively implement, and/or cause the implementation of, mapping operations, harmonization operations, toxicant related operations, toxicant mapping operations, remediation and/or mitigation operations, or the like or combinations thereof. More generally, the scope of this disclosure embraces any operating environment in which the disclosed concepts may be useful.

Example cloud computing environments, which may or may not be public, include storage environments that may provide data storage functionality for one or more clients. Some example cloud computing environments in connection with which embodiments may be employed include, but are not limited to, Microsoft Azure, Amazon AWS, Dell EMC Cloud Storage Services, and Google Cloud. More generally however, the scope of this disclosure is not limited to employment of any particular type or implementation of cloud computing environment.

In addition to the cloud environment, the operating environment may also include one or more clients that are capable of collecting, modifying, and creating, data. As such, a particular client may employ, or otherwise be associated with, one or more instances of each of one or more applications that perform such operations with respect to data. Such clients may comprise physical machines, containers, or virtual machines (VMs). Embodiments of the invention may be applications, apps, web-based, or the like.

Particularly, devices in the operating environment may take the form of software, physical machines, containers, or VMs, or any combination of these, though no particular device implementation or configuration is required for any embodiment. Similarly, data storage system components such as databases, storage servers, storage volumes (LUNs), storage disks, servers and clients, for example, may likewise take the form of software, physical machines, containers, or virtual machines (VMs), though no particular component implementation is required for any embodiment.

As used herein, the term ‘data’ is intended to be broad in scope. Further, toxicant data relates to man-made pollutions, materials, substances. Toxicant data may also relate to materials, substances, or the like that may adversely impact health or cause medical conditions.

Example embodiments are applicable to any system capable of storing and handling various types of objects, in analog, digital, or other form.

It is noted that any operation(s) of any of the methods disclosed herein, may be performed in response to, as a result of, and/or, based upon, the performance of any preceding operation(s). Correspondingly, performance of one or more operations, for example, may be a predicate or trigger to subsequent performance of one or more additional operations. Thus, for example, the various operations that may make up a method may be linked together or otherwise associated with each other by way of relations such as the examples just noted. Finally, and while it is not required, the individual operations that make up the various example methods disclosed herein are, in some embodiments, performed in the specific sequence recited in those examples. In other embodiments, the individual operations that make up a disclosed method may be performed in a sequence other than the specific sequence recited.

The embodiments disclosed herein may include the use of a special purpose or general-purpose computer including various computer hardware or software modules, as discussed in greater detail below. A computer may include a processor and computer storage media carrying instructions that, when executed by the processor and/or caused to be executed by the processor, perform any one or more of the methods disclosed herein, or any part(s) of any method disclosed.

As indicated above, embodiments within the scope of this disclosure also include computer storage media, which are physical media for carrying or having computer-executable instructions or data structures stored thereon. Such computer storage media may be any available physical media that may be accessed by a general purpose or special purpose computer.

By way of example, and not limitation, such computer storage media may comprise hardware storage such as solid state disk/device (SSD), RAM, ROM, EEPROM, CD-ROM, flash memory, phase-change memory (“PCM”), or other optical disk storage, magnetic disk storage or other magnetic storage devices, or any other hardware storage devices which may be used to store program code in the form of computer-executable instructions or data structures, which may be accessed and executed by a general-purpose or special-purpose computer system to implement the disclosed functionality. Combinations of the above should also be included within the scope of computer storage media. Such media are also examples of non-transitory storage media, and non-transitory storage media also embraces cloud-based storage systems and structures, although the scope of this disclosure is not limited to these examples of non-transitory storage media.

Computer-executable instructions comprise, for example, instructions and data which, when executed, cause a general purpose computer, special purpose computer, or special purpose processing device to perform a certain function or group of functions. As such, some embodiments may be downloadable to one or more systems or devices, for example, from a website, mesh topology, or other source. As well, the scope of this disclosure embraces any hardware system or device that comprises an instance of an application that comprises the disclosed executable instructions.

Following are some further example embodiments of the invention. These are presented only by way of example and are not intended to limit the scope of the invention in any way.

Embodiment 1. A method comprising: by a toxicant engine implemented in a computing system: retrieving toxicant data from a toxicant database in response to a query received from a user, generating a map that includes mapping layers from the toxicant data, wherein the map is adapted to account for a location included in the query and wherein the toxicant data is related to the location specified in the query, and presenting the map in a user interface.

Embodiment 2. The method of embodiment 1, further comprising generating a report from second sources including study data, traditional medicine mitigation data, alternative medicine mitigation data.

Embodiment 3. The method of embodiment 1 and/or 2, further comprising presenting the report in the user interface.

Embodiment 4. The method of embodiment 1, 2, and/or 3, wherein the report and the map are related such that selections in the map generate a change in the report based on the selections.

Embodiment 5. The method of embodiment 1, 2, 3, and/or 4, further comprising retrieving data from data sources and preparing the data to generate the toxicant data.

Embodiment 6. The method of embodiment 1, 2, 3, 4, and/or 5, wherein the data sources include data sources external to the toxicant engine.

Embodiment 7. The method of embodiment 1, 2, 3, 4, 5, and/or 6, wherein each of the mapping layers relates to a different media and/or a different toxicant.

Embodiment 8. The method of embodiment 1, 2, 3, 4, 5, 6 and/or 7, wherein the map is drillable such that specific layers can be presented in the user interface based on user selections.

Embodiment 9. The method of embodiment 1, 2, 3, 4, 5, 6, 7, and/or 8, wherein the user interface is configured to adjust the map to show individual layers based on media, individual layers based on toxicant, sets of layers based on media, sets of layers based on toxicant, individual layers based on location, sets of layers based on locations, or combinations thereof.

Embodiment 10. The method of embodiment 1, 2, 3, 4, 5, 6, 7, 8, and/or 9, wherein the media is one or more of air, soil, water, water tables, or the like.

Embodiment 11. The method of embodiment 1, 2, 3, 4, 5, 6, 7, 8, 9, and/or 10, further comprising updating the map in real-time and updating the map to account for toxicant spread, time, weather, or combinations thereof.

Embodiment 12. The method of embodiment 1, 2, 3, 4, 5, 6, 7, 8, 9, 10, and/or 11, further comprising presenting a score in the user interface representing a risk to the user.

Embodiment 13. The method of embodiment 1, 2, 3, 4, 5, 6, 7, 8, 9, 10, 11, and/or 12, wherein the risk is based on a location of the user and a location of the toxicant.

Embodiment 14. The method of embodiment 1, 2, 3, 4, 5, 6, 7, 8, 9, 10, 11, 12, and/or 13, wherein the risk includes multiple risks, each associated with a different toxicant.

Embodiment 15. The method of embodiment 1, 2, 3, 4, 5, 6, 7, 8, 9, 10, 11, 12, 13, and/or 14, further comprising determining a trust level for the map and for the risk based on crowd sourced input.

Embodiment 16. The method of embodiment 1, 2, 3, 4, 5, 6, 7, 8, 9, 10, 11, 12, 13, 14, and/or 15, wherein the map accounts for regulations, time, planned mitigations, or combinations thereof.

Embodiment 17. The method of embodiment 1, 2, 3, 4, 5, 6, 7, 8, 9, 10, 11, 12, 13, 14, 15, and/or 16, further comprising providing a map that is dynamic and accounts for a history of locations, multiple locations, time, or combinations thereof.

Embodiment 18. A computing system for performing any of the operations, methods, or processes, or any portion of any of these, disclosed herein.

Embodiment 19. A non-transitory storage medium or storage device having stored therein instructions that are executable by one or more hardware processors to perform operations comprising the operations of any one or more of embodiments 1-17.

Although the subject matter has been described in language specific to structural features and/or methodological acts, it is to be understood that the subject matter defined in the appended claims is not necessarily limited to the specific features or acts described above. Rather, the specific features and acts disclosed herein are disclosed as example forms of implementing the claims.

As used herein, the term module, component, client, agent, service, engine, or the like may refer to software objects or routines that execute on the computing system. These may be implemented as objects or processes that execute on the computing system, for example, as separate threads. While the system and methods described herein may be implemented in software, implementations in hardware or a combination of software and hardware are also possible and contemplated. In the present disclosure, a ‘computing entity’ may be any computing system as previously defined herein, or any module or combination of modules running on a computing system.

In at least some instances, a hardware processor is provided that is operable to carry out executable instructions for performing a method or process, such as the methods and processes disclosed herein. The hardware processor may or may not comprise an element of other hardware, such as the computing devices and systems disclosed herein.

In terms of computing environments, embodiments may be performed in client-server environments, whether network or local environments, or in any other suitable environment. Suitable operating environments for at least some embodiments include cloud computing environments where one or more of a client, server, or other machine may reside and operate in a cloud environment.

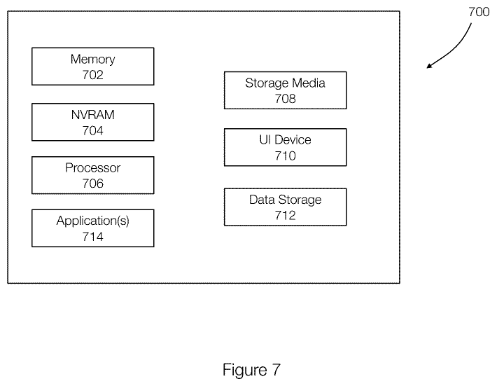

With reference briefly now to FIG. 7, any one or more of the entities disclosed, or implied, by the Figures, and/or elsewhere herein, may take the form of, or include, or be implemented on, or hosted by, a physical computing device, one example of which is denoted at 700. As well, where any of the aforementioned elements comprise or consist of a virtual machine (VM), that VM may constitute a virtualization of any combination of the physical components disclosed in FIG. 7.

In the example of FIG. 7, the physical computing device 700 includes a memory 702 which may include one, some, or all, of random access memory (RAM), non-volatile memory (NVM) 704 such as NVRAM for example, read-only memory (ROM), and persistent memory, one or more hardware processors 706, non-transitory storage media 708, UI device 710, and data storage 712. One or more of the memory components 702 of the physical computing device 700 may take the form of solid state device (SSD) storage. As well, one or more applications 700 may be provided that comprise instructions executable by one or more hardware processors 706 to perform any of the operations, or portions thereof, disclosed herein. The device 700 is also representative of a cloud environment, server clusters, edge-based systems, or the like or combinations thereof.

Such executable instructions may take various forms including, for example, instructions executable to perform any method or portion thereof disclosed herein, and/or executable by/at any of a storage site, whether on-premises at an enterprise, or a cloud computing site, client, datacenter, data protection site including a cloud storage site, or backup server, to perform any of the functions disclosed herein. As well, such instructions may be executable to perform any of the other operations and methods, and any portions thereof, disclosed herein.

The described embodiments are to be considered in all respects only as illustrative and not restrictive. All changes which come within the meaning and range of equivalency of the claims are to be embraced within their scope.

What is claimed is:

1. A method comprising:
by a toxicant engine implemented in a computing system:
retrieving toxicant data from a toxicant database in response to a query received from a user;
generating a map that includes mapping layers from the toxicant data, wherein the map is adapted to account for a location included in the query and wherein the toxicant data is related to the location specified in the query; and
presenting the map in a user interface.

2. The method of claim 1, further comprising generating a report from second sources including study data, traditional medicine mitigation data, alternative medicine mitigation data.

3. The method of claim 2, further comprising presenting the report in the user interface.

4. The method of claim 3, wherein the report and the map are related such that selections in the map generate a change in the report based on the selections.

5. The method of claim 1, further comprising retrieving data from data sources and preparing the data to generate the toxicant data, wherein the data sources include one or more of data sources internal to the toxicant engine and data sources external to the toxicant engine.

6. The method of claim 1, wherein the user interface comprises an address comparison dashboard, wherein the address comparison dashboard illustrates toxin intensity ratings and specific toxicants for multiple addresses selected by a user and wherein the toxicants are identified by media and/or classification.

7. The method of claim 1, wherein each of the mapping layers relates to a different media and/or a different toxicant, wherein the map is drillable such that specific layers can be presented in the user interface based on user selections.

8. The method of claim 1, wherein further comprising classifying the toxicants based on their impact on human health.

9. The method of claim 1, wherein the user interface is configured to adjust the map to show individual layers based on media, individual layers based on toxicant, sets of layers based on media, sets of layers based on toxicant, individual layers based on location, sets of layers based on locations, or combinations thereof.

10. The method of claim 1, wherein the media is one or more of air, soil, water, water tables, and/or nuclear, a physical structure, or combinations thereof.

11. The method of claim 1, further comprising updating the map in real-time and updating the map to account for toxicant spread, time, weather, climate events, or combinations thereof, wherein the user interface is further configured to illustrate an estimate of future toxicant spread and/or toxicant exposure.

12. The method of claim 1, further comprising presenting a score in the user interface representing a risk to the user.

13. The method of claim 12, wherein the risk is based on a location of the user and a location of the toxicant.

14. The method of claim 12, wherein the risk includes multiple risks, each associated with a different toxicant or a different classification of toxicants or based on an impact on human health.

15. The method of claim 12, further comprising determining a trust level for the map and for the risk based on crowd sourced input.

16. The method of claim 12, wherein the map accounts for regulations, time, planned mitigations, or combinations thereof.

17. The method of claim 12, further comprising providing a map that is dynamic and accounts for a history of locations, multiple locations, time, or combinations thereof.

18. One or more hardware storage devices that store instructions that are executable by one or more processors of a computer system to cause the computer system to perform operations including:
retrieving toxicant data from a toxicant database in response to a query received from a user;
generating a map that includes mapping layers from the toxicant data, wherein the map is adapted to account for a location included in the query and wherein the toxicant data is related to the location specified in the query; and
presenting the map in a user interface.

19. The hardware storage of claim 18, the operations further comprising:
generating a report from second sources including study data, traditional medicine mitigation data, alternative medicine mitigation data;
presenting the report in the user interface, wherein the report and the map are related such that selections in the map generate a change in the report based on the selections;
retrieving data from data sources and preparing the data to generate the toxicant data, wherein the data sources include data sources external to the toxicant engine, wherein each of the mapping layers relates to a different media and/or a different toxicant, wherein the map is drillable such that specific layers can be presented in the user interface based on user selections, wherein the user interface is configured to adjust the map to show individual layers based on media, individual layers based on toxicant, sets of layers based on media, sets of layers based on toxicant, individual layers based on location, sets of layers based on locations, or combinations thereof.

20. The hardware storage of claim 18, the operations further comprising:
updating the map in real-time and updating the map to account for toxicant spread, time, weather, or combinations thereof;
presenting a score in the user interface representing a risk to the user, wherein the risk is based on a location of the user and a location of the toxicant, wherein the risk includes multiple risks, each associated with a different toxicant;
determining a trust level for the map and for the risk based on crowd sourced input;
wherein the map accounts for regulations, time, planned mitigations, or combinations thereof, and
wherein the map is dynamic and accounts for a history of locations, multiple locations, time, or combinations thereof.
